(19) United States
(12) Patent Application Publication
(10) Pub. No.: US 2010/0190797 A1
(43) Pub. Date: Jul. 29, 2010

(54) CRYSTALLINE POLYMOPHIC FORMS OF ZOPICLONE, PROCESSES FOR THEIR PREPARATION AND THEIR PHARMACEUTICAL COMPOSITIONS

(75) Inventors: Dharmaraj Ramchandra Rao, Mumbai (IN); Rajendra Narayanrao Kankan, Mumbai (IN); Maruti Ganpati Ghaghare, Thane (IN); Sunilkumar Saroj, Mumbai (IN)

Correspondence Address: VOLPE AND KOENIG, P.C., UNITED PLAZA, SUITE 1600, 30 SOUTH 17TH STREET, PHILADELPHIA, PA, 19103, US

(73) Assignee: CIPLA LTD., Mumbai (IN)

(21) Appl. No.: 12/664,949

(22) PCT Filed: Dec. 10, 2008
(86) PCT No.: PCT/IN2008/000829

§ 371 Date: Dec. 16, 2009

(30) Foreign Application Priority Data
Dec. 11, 2007 (IN) 2423/MUM/2007

(51) Int. Cl. A61K 31/4985; C07D 487/04

(52) U.S. Cl. 514/249; 544/350

(57) ABSTRACT

Disclosed are processes for the preparation of crystalline polymorphic forms of zopiclone and their pharmaceutical compositions for use as a sedative.

### FIELD OF THE INVENTION

The present invention relates to a process for preparation of highly pure Zopiclone. This invention also relates to polymorphs of Zopiclone, processes for their preparation and pharmaceutical compositions thereof.

### BACKGROUND OF THE INVENTION

Zopiclone is a sedative hypnotic agent. It is a highly potent drug substance used in treatment of sleep disorders such as insomnia and convulsive disorders such as epilepsy.

The process for its preparation as disclosed in U.S. Pat. No. 3,862,149 comprises 1-chlorocarbonyl-4-methyl-piperazine as the precursor to get 6-(5-chloro-2-pyridinyl)-5-hydroxy-7-oxo-5,6-dihydropyrrolo[3,4-b]pyrazine which on reaction with phenyl chloroformate followed by 3-amino-6-methylpyridazine yields Zopiclone.

By following the process of U.S. Pat. No. '149, Zopiclone obtained contains some impurities, which results in less yield and purity of the product. There is a necessity for a process of purification of Zopiclone to get highly pure product without affecting the yields. The process should be simple involving less number of steps and readily available reagents. Further, there is no teaching in the U.S. Pat. No. '149 about the polymorphism of Zopiclone nor there is any characterization data available for the product obtained by the process of U.S. Pat. No. '149.

A drug substance may exist in different polymorphic forms with substantially difference in certain properties such as particle size, hardness, glass transition temperatures, stability and solubility that may be quite important for pharmacological action of the drug substance.

A research paper published by Terblanche Et. al, Drug Development and Industrial Pharmacy, 26(5), 531-537 (2000) describes three crystalline polymorphs of Zopiclone viz. an anhydrous Form A, a dihydrate Form B and a mixture of both the Forms A and B and their effect on the physicochemical properties of Zopiclone. All these forms are characterized by X-ray diffraction, Differential scanning calorimetry (DSC), Infra-red spectrophotometry and particle size. Zopiclone obtained by the process of U.S. Pat. No. '419 has the characteristics of Form A.

Zopiclone Form A is found to be unstable and gets converted into a thermodynamically stable dihydrate Form B.

In this context, the present invention provides new process for preparation of highly pure Zopiclone, new polymorphs of Zopiclone and pharmaceutical compositions thereof.

### OBJECT OF THE INVENTION

It is an object of the present invention to provide a process for preparation of Zopiclone having purity greater than 99.5%.

It is another object of the present invention to provide polymorphs of Zopiclone hereinafter referred to as Form C and Form D.

It is yet another object of the present invention to provide a pharmaceutical composition comprising polymorphs of Zopiclone and one or more pharmaceutically acceptable carriers.

### SUMMARY OF THE INVENTION

According to first aspect of the invention there is provided Zopiclone of purity of at least 99.5%.

According to an aspect of the present invention there is provided a process for the preparation of Zopiclone comprising the steps of:
i. Coupling of 6-(5-chloropyrid-2-yl)-5-hydroxy-7-oxo-5,6-di-hydropyrrolo[3,4,b]pyrazine with phenyl chloroformate in presence of anhydrous pyridine to give 6-(5-chloropyrid-2-yl)-5-phenoxycarbonyloxy-7-oxo-5,6-di-hydropyrrolo[3,4,b]pyrazine;
ii. Reaction of 6-(5-chloropyrid-2-yl)-5-phenoxycarbonyloxy-7-oxo-5,6-di-hydropyrrolo[3,4,b]pyrazine with 1-methylpiprazine in presence of a suitable organic solvent to yield Zopiclone.

According to another aspect of the present invention there is provided a new crystalline form of Zopiclone designated as Form C which is characterized by having an X-ray powder diffraction pattern that comprises peaks with 2theta values (±0.2) of at least 5.47, 5.80, 6.25, 10.31, 12.47, 13.50, 13.84, 15.68, 15.72, 16.12, 16.39, 18.79, 19.77, 20.24, 20.38, 20.47, 23.70, 23.80, 24.78, 24.81, 24.93, 25.69, 26.79, 26.90, 27.59, 27.76, 27.87, 28.10, 29.22, 29.29, 31.01, 31.12, 31.40, 31.48, 33.18, 35.50, 35.78.

In an embodiment, Zopiclone Form C is characterised as having an XRPD pattern with peaks as shown in Table 1 below.

In an embodiment, Zopiclone Form C is characterised as having an XRPD pattern as shown in FIG. 1.

Zopiclone Form C may be also characterized by Infra-red (IR) absorption spectrophotometry having characteristic peaks at 3496, 3388, 2973, 2937, 2850, 2794 cm⁻¹ in the IR spectrum.

In an embodiment, Zopiclone Form C is characterized by having an IR spectrum as shown in FIG. 2.

According to another aspect of the present invention there is provided a process for Zopiclone Form C comprising the steps of:
i. Stirring Zopiclone in a suitable organic solvent followed by filtration, washing and concentration to get a residue;
ii. Stirring of the residue of step (i) in diisopropyl ether followed by filtration;
iii. Dissolution of solid from step (ii) in dichloromethane and filtration of the insolubles;
iv. Washing of the organic layer with alkali solution followed by water and partial concentration under vacuum;
v. Addition of isopropyl alcohol to the above and further concentration to get a residue;
vi. Addition of a mixture of acetonitrile and diisopropyl ether to (v) and filtration of the resulting solid;
vii. Dissolution of solid from (vi) in N,N-dimethylformamide by heating at a temperature of 50-80° C. to get a clear solution followed by charcoalization;
viii. Heating of the clear filtrate to 50-80° C. and addition of water at the same temperature to precipitate the solid followed by gradual cooling of the resulting suspension;
ix. Filtration of the solid from (viii) followed by washing with a solvent or a mixture of solvents such as N,N-dimethylformamide and water and further drying to get crystalline Zopiclone Form C.

Zopiclone Form C prepared by the process may be in the form as described above.

According to another aspect of the present invention there is provided a new crystalline anhydrous form of Zopiclone designated as Form D.

In an embodiment, Crystalline Form D of Zopiclone is characterized by having an X-ray powder diffraction pattern that comprises peaks with 2theta values (±0.2) of at least 9.34, 9.95, 10.14, 10.94, 11.24, 12.03, 12.40, 12.65, 14.87, 15.48, 15.92, 16.77, 17.18, 19.82, 20.12, 20.68, 21.34, 22.35, 22.77, 23.05, 24.78, 25.40, 25.64, 26.91, 27.38, 28.26, 28.54, 28.69, 30.25, 30.37, 30.69, 31.08, 31.59, 33.22, 33.71, 34.33, 35.02, 35.91, 36.29, 37.88, 38.00.

In an embodiment, Zopiclone Form D is characterised as having an XRPD pattern with peaks as shown in Table 2 below.

In an embodiment, Zopiclone Form D is characterised as having an XRPD pattern as shown in FIG. 3.

Zopiclone Form D may be also characterized by Infra-red (IR) absorption spectrophotometry having characteristic peaks at 3109, 3077, 2973, 2929, 2848, 2797, 2747 cm⁻¹ in the IR spectrum.

In an embodiment, Zopiclone Form D is characterized by having an IR spectrum as shown in FIG. 4.

According to another aspect of the present invention there is provided a process for Zopiclone Form D comprising the steps of:
i. Reaction of 6-(5-chloropyrid-2-yl)-5-phenoxycarbonyloxy-7-oxo-5,6-di-hydropyrrolo[3,4,b]pyrazine with 1-methylpiperazine in presence of a suitable organic solvent at a temperature of 5-20° C.;
ii. Addition of water to the reaction mass after cooling to 0-10° C. followed by filtration of the resulting solid;
iii. Dissolution of the solid from (ii) in a suitable organic solvent and filtration of the insolubles;
iv. The filtrate from (iii) is washed with an alkali solution followed by water and partial concentration under vacuum;
v. Addition of isopropyl alcohol and further concentration to get a slurry;
vi. Filtration of the slurry from (v) and washing with a suitable solvent or a mixture of solvents followed by drying to get crystalline Zopiclone Form D.

Zopiclone Form D of the present invention is stable and non-hygroscopic as it does not capture moisture even on exposure to air.

According to another aspect of the present invention, there is provided pharmaceutical compositions comprising forms C and D of Zopiclone as described above, together with one or more pharmaceutically acceptable excipients.

### BRIEF DESCRIPTION OF THE DRAWINGS

FIG. 1 depicts an X-ray diffraction spectrum of Zopiclone Form C.

FIG. 1A shows the 2θ value of FIG. 1.

FIG. 2 depicts an Infra-red absorption spectrum of Zopiclone Form C.

FIG. 3 depicts an X-ray diffraction spectrum of Zopiclone Form D.

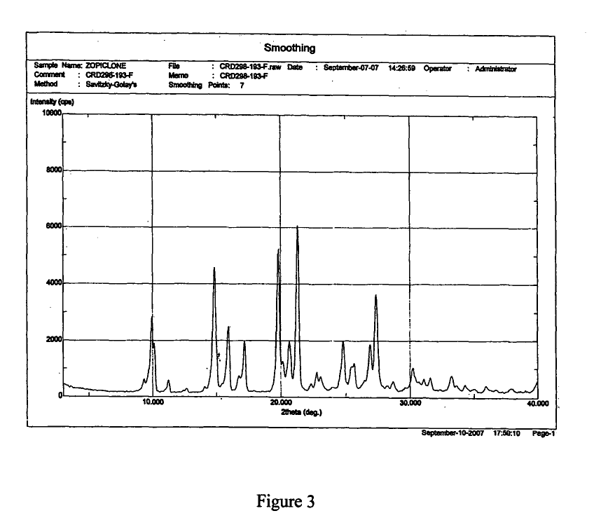

FIG. 3A shows the 2θ value of FIG. 3.

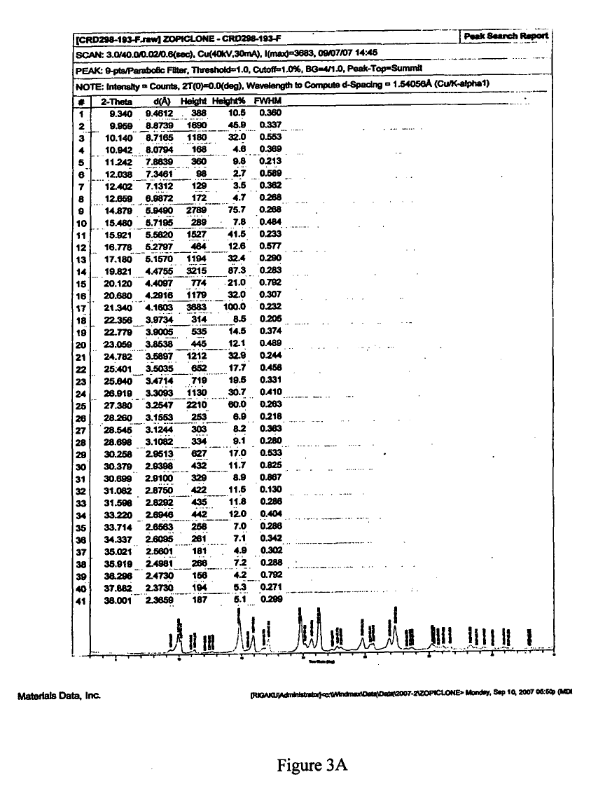

FIG. 4 depicts an Infra-red absorption spectrum of Zopiclone Form D.

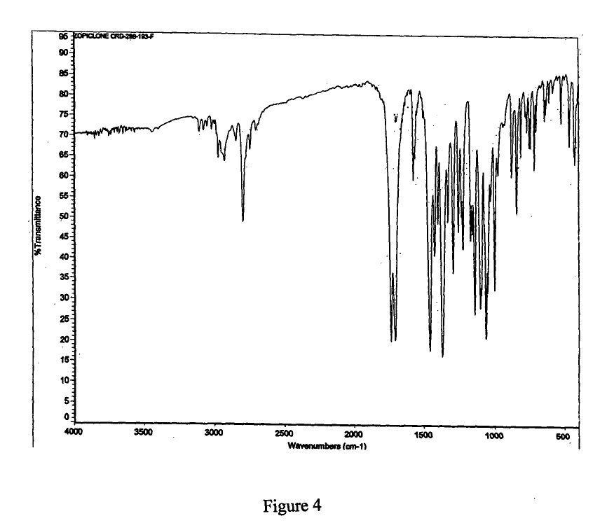

### DETAILED DESCRIPTION OF THE INVENTION

Zopiclone is prepared by the following method using commercially available 6-(5-chloropyrid-2-yl)-5-hydroxy-7-oxo-5,6-di-hydropyrrolo[3,4,b]pyrazine as the precursor. The process can be described as follows:
a) coupling of 6-(5-chloropyrid-2-yl)-5-hydroxy-7-oxo-5,6-di-hydropyrrolo[3,4,b]pyrazine with phenyl chloroformate in presence of anhydrous pyridine to give 6-(5-chloropyrid-2-yl)-5-phenoxycarbonyloxy-7-oxo-5,6-di-hydropyrrolo[3,4,b]pyrazine;
b) 6-(5-chloropyrid-2-yl)-5-phenoxycarbonyloxy-7-oxo-5,6-di-hydropyrrolo[3,4,b]pyrazine on further reaction with 1-methylpiprazine in presence of a suitable solvent such as acetone, acetonitrile, N,N-dimethylformamide, ethyl acetate, most preferably acetonitrile yields Zopiclone which is subjected to purification and this forms another aspect of the present invention.

In this aspect, Zopiclone is stirred in a suitable solvent such as acetone, acetonitrile, ethylene dichloride, halogenated solvents such as dichloromethane, chloroform, more preferably, acetonitrile, filtered, washed with the same solvent and concentrated to get a residue. The residue is stirred in diisopropyl ether and resulting solid is filtered. Solid is further dissolved in dichloromethane and the insolubles are filtered. The organic layer is washed with alkali solution, preferably sodium hydroxide solution followed by water. Further the organic layer is separated and partially concentrated under vacuum. To this, isopropyl alcohol is added and further concentrated to get a residue. A mixture of acetonitrile and diisopropyl ether is added and stirred. The resulting solid is filtered and dried.

The solid is dissolved in N,N-dimethylformamide by heating at a temperature of 50-80° C., preferably at 60-65° C. to get a clear solution and charcoalised. The clear filtrate is heated to 50-80° C., preferably at 60-65° C. and water is added thereto at the same temperature to precipitate the solid. The resulting suspension is cooled gradually to a temperature of 25-30° C.

The solid is filtered and washed with a solvent or a mixture of solvents such as N,N-dimethylformamide and water. Further the solid is dried at a temperature of 85-95° C. to get crystalline Zopidlone (HPLC purity: 99.9%).

Zopiclone, obtained by the process of this invention, is in pure form and is free of inorganic impurities and all other impurities. Surprisingly, we found that Zopiclone obtained by the above process is of a new polymorphic form. It is characterized and designated as Zopiclone Form C.

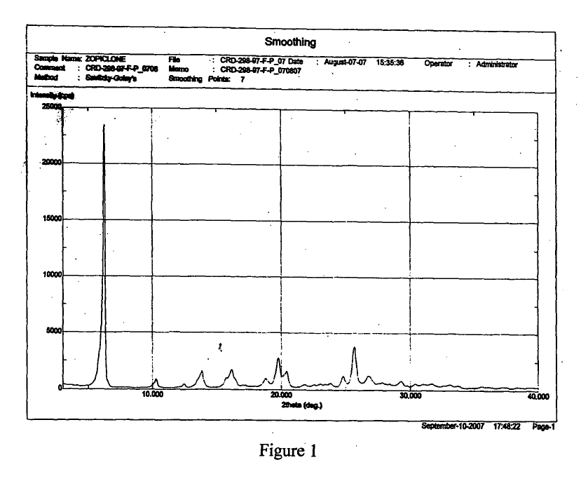

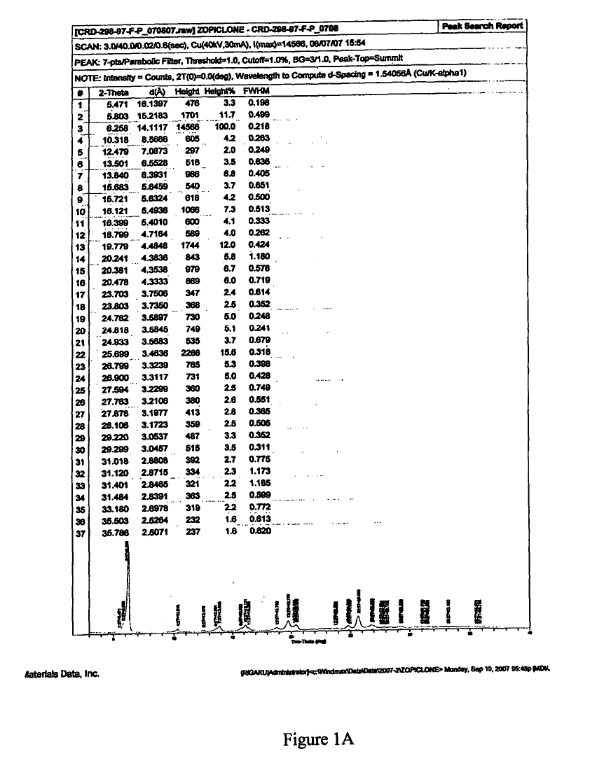

In yet another aspect, novel Crystalline Form C of Zopiclone of the present invention is characterized by X-ray powder diffraction (XRPD) pattern shown in FIG. 1. The XRPD of Zopiclone Form C was measured on Rigaku miniflex advance powder X-ray diffractometer using copper-K_{α-1} radiation source. The characteristic XRPD spectrum of Zopiclone Form C characterized by having the peaks at least as tabulated in the Table 1 below:

TABLE 1
Zopiclone Form ‘C’

| Diffraction angles | Relative intensity |
|---|---|
| (2θ°) | (% I/Io) |
| 5.47 | 3.3 |
| 5.80 | 11.7 |
| 6.25 | 100 |
| 10.31 | 4.2 |
| 12.47 | 2.0 |
| 13.50 | 3.5 |
| 13.84 | 6.8 |
| 15.68 | 3.7 |
| 15.72 | 4.2 |
| 16.12 | 7.3 |
| 16.39 | 4.1 |
| 18.79 | 4.0 |
| 19.77 | 12.0 |
| 20.24 | 5.8 |
| 20.38 | 6.7 |
| 20.47 | 6.0 |
| 23.70 | 2.4 |
| 23.80 | 2.5 |
| 24.78 | 5.0 |
| 24.81 | 5.1 |
| 24.93 | 3.7 |
| 25.69 | 15.6 |
| 26.79 | 5.3 |
| 26.90 | 5.0 |
| 27.59 | 2.5 |
| 27.76 | 2.6 |
| 27.87 | 2.8 |
| 28.10 | 2.5 |
| 29.22 | 3.3 |
| 29.29 | 3.5 |
| 31.01 | 2.7 |
| 31.12 | 2.3 |
| 31.40 | 2.2 |
| 31.48 | 2.5 |
| 33.18 | 2.2 |
| 35.50 | 1.6 |
| 35.78 | 1.6 |

Zopiclone Form C of the present invention is characterized by Infra-red (IR) absorption spectrophotometry having characteristic peaks at 3496, 3388, 2973, 2937, 2850, 2794 cm⁻¹ in the IR spectrum.

Form C is also found to be thermodynamically unstable and gets converted to more stable hydrate form of Zopiclone.

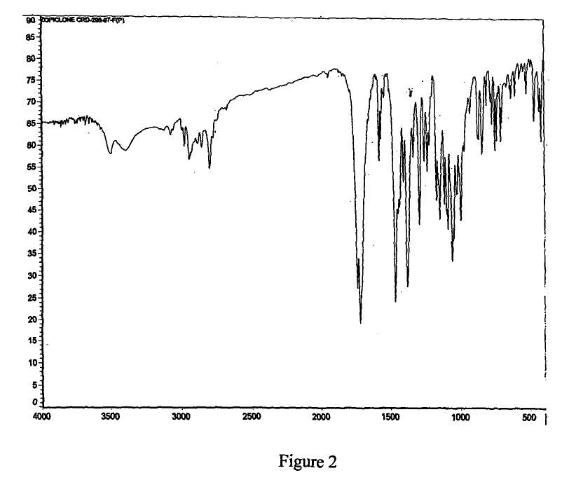

In yet another preferred aspect, the present invention provides a stable, non-hygroscopic, anhydrous crystalline Zopiclone Form D. It is characterized by X-ray powder diffraction, and Infra-red absorption spectrophotometry. The X-ray powder diffraction (XRPD) pattern is as depicted in FIG. 2. The XRPD of Zopiclone Form D was measured on Rigaku miniflex advance powder X-ray diffractometer using copper-K_{α-1} radiation source. The characteristic XRPD spectrum of Zopiclone Form D characterized by having the peaks at least as tabulated in the Table 2 below:

TABLE 2
Zopiclone Form ‘D’

| Diffraction angles | Relative intensity |
|---|---|
| (2θ°) | (% I/Io) |
| 9.34 | 10.5 |
| 9.95 | 45.9 |
| 10.14 | 32.0 |
| 10.94 | 4.6 |
| 11.24 | 9.8 |
| 12.03 | 2.7 |
| 12.40 | 3.5 |
| 12.65 | 4.7 |
| 14.87 | 75.7 |
| 15.48 | 7.8 |
| 15.92 | 41.5 |
| 16.77 | 12.6 |
| 17.18 | 32.4 |
| 19.82 | 87.3 |
| 20.12 | 21.0 |
| 20.68 | 32.0 |
| 21.34 | 100.0 |
| 22.35 | 8.5 |
| 22.77 | 14.5 |
| 23.05 | 12.1 |
| 24.78 | 32.9 |
| 25.40 | 17.7 |
| 25.64 | 19.5 |
| 26.91 | 30.7 |
| 27.38 | 60.0 |
| 28.26 | 6.9 |
| 28.54 | 8.2 |
| 28.69 | 9.1 |
| 30.25 | 17.0 |
| 30.37 | 11.7 |
| 30.69 | 8.9 |
| 31.08 | 11.5 |
| 31.59 | 11.8 |
| 33.22 | 12.0 |
| 33.71 | 7.0 |
| 34.33 | 7.1 |
| 35.02 | 4.9 |
| 35.91 | 7.2 |
| 36.29 | 4.2 |
| 37.88 | 5.3 |
| 38.00 | 5.1 |

Zopiclone Form D of the present invention is characterized by Infra-red (IR) absorption spectrophotometry having characteristic peaks at 3109, 3077, 2973, 2929, 2848, 2797, 2747 cm⁻¹ in the IR spectrum.

The characterization of both the crystalline forms, C and D, of the present invention clearly distinguishes from the other known polymorphs of Zopiclone.

In one more aspect, the present invention provides a process for preparation of Zopiclone Form D which comprises;

Reacting 6-(5-chloropyrid-2-yl)-5-phenoxycarbonyloxy-7-oxo-5,6-di-hydropyrrolo[3,4,b]pyrazine with 1-methylpiperazine in presence of a suitable solvent such as acetone, acetonitrile, N,N-dimethylformamide, ethyl acetate most preferably N,N-dimethylformamide at a temperature of 5-20° C., the reaction mass is further cooled to 0-10° C. Water is added under stirring and the resulting solid is filtered. The filtered solid is dissolved in a suitable solvent such as acetonitrile, acetone, ethylene dichloride, dichloromethane, chloroform, most preferably, dichloromethane and the insolubles are filtered.

The clear filtrate is washed with an alkali solution, preferably sodium hydroxide solution followed by water. Further, the organic layer is separated and concentrated partially under vacuum. To this isopropyl alcohol is added and further concentrated to get slurry. This slurry is stirred, filtered and washed with a suitable solvent or a mixture of solvents. The mixture of solvents is more particularly, a mixture of acetonitrile and diisopropyl ether. The solid is dried at a temperature of 85-95° C., most preferably at 90° C., to get crystalline Zopiclone Form D.

In one more aspect the present invention includes, within the scope of the present invention, pharmaceutical compositions comprising one of the polymorphs of Zopiclone that can be admixed with one or more pharmaceutical carriers. The pharmaceutical composition may be, but are non-limiting to, oral dosage form such as liquids or suspensions, emulsions or in solid dosage forms such as tablets, capsules, powders, granules or inhalation formulations such as aerosols or injectables or parenteral dosage forms, transdermal administrations and the like.

There follow, by way of non-restrictive explanation of the present invention, the following examples.

### EXAMPLES

### Example 1

Dichloromethane (400 ml) was charged in a reaction vessel. 6-(5-chloropyrid-2-yl)-5-hydroxy-7-oxo-5,6-dihydropyrrolo-[3,4,b]pyrazine (100 gms) was added thereto under stirring. The reaction mass was cooled to 0° C. and phenyl chloroformate (72 ml) diluted in dichloromethane (200 ml) was added dropwise in 1-1.5 hours maintaining temperature of 0-5° C. The reaction temperature was gradually raised to 25-30° C. and stirred for 2-2.5 hrs. The reaction mass was cooled to 0-5° C. and ice cold water (500 ml) was added. This was stirred for 30-45 minutes at 0-10° C. The solid obtained was filtered and washed with chilled water (100 ml) followed by acetonitrile (70 ml) and dried. The solid was stirred in diisopropyl ether (400 ml) at 25-30° C. and filtered. Washings were given with diisopropyl ether (100 ml) and dried at 60-70° C.

Yield=125 gms

### Example 2

a) The compound (100 gms) obtained from Example 1 was charged to a vessel. Acetonitrile (1000 ml) was added under argon at 25-30° C. N-methyl piperazine (60 ml) was added dropwise at a temperature of 15-20° C. and stirred for 3-4 hours at 15-20° C. The reaction mixture was filtered and washed with diisopropyl ether (30 ml). This was concentrated at 25-30° C. and stirred with diisopropyl ether (200 ml). This was filtered, washed with diisopropyl ether (2×25 ml) and dried to get a solid.

Yield=87 gms.

b) To remove insoluble material, product obtained from step a) was treated with dichloromethane (700 ml) at room temperature. The insoluble material was filtered off and dichloromethane layer was washed with 5% sodium hydroxide solution (3×100 ml) followed by water (2×500 ml) and dried over sodium sulphate. This was concentrated to a minimum volume of dichloromethane and stripped with isopropyl alcohol (3×100 ml). The solid was isolated in a mixture of acetonitrile and diisopropyl ether (5:95) and suck dried.

Yield=55 gms.

### Example 3

55 gms of the product obtained from step b) of example 2 was dissolved in N,N-dimethylformamide (220 ml) and heated at a temperature of 60-65° C. to get a clear solution. This solution was treated with 5.5 gms of charcoal and heated at 60-65° C. for at least 30 minutes. This was filtered hot over hyflo and washed with N,N-dimethylformamide (55 ml). The clear filtrate was heated at a temperature of 60-65° C. and water (275 ml) was added in 30 minutes to get a solid. The solid was cooled gradually to 25-30° C., filtered and washed with 1:1 mixture of N,N-dimethylformamide and water (2×25 ml). This was finally washed with water (3×200 ml) and dried at 90-93° C. to get crystalline Zopiclone.

Yield=51 gms.

### Example 4

a) 20 gms of compound obtained from Example 1 was charged to a vessel. N,N-dimethylformamide (80 ml) was added under argon and cooled to 10-15° C. N-methyl piperazine (9 ml) was added dropwise at a temperature of 10-15° C. in 15-30 minutes and stirred for 3 hours. The reaction mass was cooled to 0-5° C. followed by addition of water (80-ml) maintaining temperature of 0-5° C. The reaction mass was stirred at a temperature of 30° C. for one hour and filtered. Filtered reaction mass was washed with water (2×100 ml) and suck dried.

b) 38 gms of the product obtained from step a) was treated with dichloromethane (140 ml) and stirred for 30 minutes. The insolubles were filtered and organic layer washed with a minimum volume of dichloromethane. The organic layer was washed with 5% sodium hydroxide solution (3×20 ml) followed by water (4×100 ml) and dried over sodium sulphate. This was concentrated to get a minimum volume of dichloromethane and stripped with (3×20 ml) of isopropyl alcohol to get a slurry. The slurry was stirred with acetonitrile (8 ml) for 15 minutes and then 152 ml of diisopropyl ether was added. The above slurry was stirred for one hour at 25-30° C. The reaction mass was filtered and washed with 5% mixture of acetonitrile and diisopropyl ether (5:95) and dried at a temperature of 90-95° C. under vacuum.

Yield=11.0-11.5 gms.

1. A process for the preparation of crystalline Zopiclone comprising the steps of:
i) suspending zopiclone in a mixture of acetonitrile and diisopropyl ether, filtering and drying the resulting solid;
ii) dissolving the solid in step i) in N,N-dimethylformamide to get a clear solution;
iii) heating and then precipitating solid; and
iv) filtering and drying the solid to get crystalline zopiclone.

2. A process according to step i) of claim 1, wherein the Zopiclone is free of insoluble impurities.

3. A process according to claim 1, wherein the crystalline Zopiclone formed has purity of at least 99.5%.

4. A composition comprising crystalline zopiclone Form C.

5. The composition of claim 4, wherein the Form C has an X-ray powder diffraction pattern that comprises peaks with 2theta values of at least 5.8, 6.2, 16.1, 19.7 and 25.6 ° 2Θ±0.2 ° 2Θ.

6. The composition of claim 4, wherein the Zopiclone Form C has an X-ray powder diffraction pattern comprising peaks at 10.3, 12.4, 13.5, 13.8, 16.3 and 18.7 ° 2Θ±0.2 ° 2θ.

7. The composition of claim 4, wherein the Zopiclone Form C has an X-ray powder diffraction pattern comprising peaks at 5.8, 6.2, 10.3, 12.4, 13.5, 13.8, 16.1, 16.3, 18.7 and 19.7° 2Θ±0.2 ° 2Θ.

8. (canceled)

9. The composition of claim 4, wherein the Zopiclone Form C has an Infra-red absorption spectrum (IR) comprising characteristic peaks at about 3496, 3388, 2973, 2937, 2850, and 2794 cm⁻¹.

10. (canceled)

11. A composition comprising crystalline Zopiclone Form D having an X-ray powder diffraction pattern that comprises peaks with 2theta values of at least 14.8, 19.8, 21.3, 27.3 ° 2Θ±0.2 ° 2Θ.

12. The composition of claim 11, wherein the Zopiclone Form D has an X-ray powder diffraction pattern comprising peaks at 9.9, 10.1, 14.8, 15.9, 17.1, 19.8, 20.6, 21.3, 24.7, 26.9 and 27.3 ° 2Θ±0.2 ° 2Θ.

13. (canceled)

14. The composition of claim 11, wherein the Zopiclone Form D has an Infra-red absorption spectrum comprising characteristic peaks at about 3109, 3077, 2973, 2929, 2848, 2797, 2747 cm⁻¹.

15. (canceled)

16. A process for preparation of crystalline Zopiclone Form D comprising steps of:
i) dissolving Zopiclone in N,N-dimethylformamide and filtering;
ii) washing clear filtrate with an alkali solution;
iii) separating and concentrating the organic layer, adding isopropyl alcohol thereto and concentrating further to get a slurry; and
iv) filtering the slurry and drying the resultant solid to get crystalline
Zopiclone form D.

17. A pharmaceutical composition comprising crystalline forms of Zopiclone as claimed in any of claims 1, 4 and 9 together with one or more pharmaceutically acceptable excipients.

18-20. (canceled)
